(19) United States
(12) Patent Application Publication
(10) Pub. No.: US 2013/0047993 A1
(43) Pub. Date: Feb. 28, 2013

(54) TRACHEAL TUBE HAVING A FLANGE WITH A VARIABLE VOLUME

(71) Applicant: Olaf Lally, Galway (IE)

(72) Inventor: Olaf Lally, Galway (IE)

(73) Assignee: Nellcor Puritan Bennett LLC, Boulder, CO (US)

(21) Appl. No.: 13/222,567

(22) Filed: Aug. 31, 2011

(51) Int. Cl. A61M 16/04

(52) U.S. Cl. 128/207.15; 128/207.14

(57) ABSTRACT

Various embodiments of tracheostomy tube assemblies including a flange member having a variable volume are provided. The tracheostomy tube assemblies may include a cannula having a distal end and a proximal end. The distal end may be adapted to be inserted into a patient's trachea, and the flange member may be disposed about the proximal end of the cannula. The flange member is adapted to expand to a first volume and to contract to a second volume, the first volume being greater than the second volume.

### BACKGROUND

The present disclosure relates generally to medical devices and, more particularly, to airway devices, such as tracheostomy tubes.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the course of treating a patient, a tube or other medical device may be used to control the flow of air, food, fluids, or other substances into the patient. For example, medical devices, such as tracheal tubes, may be used to control the flow of air and medicaments into or out of a patient's airway. In many instances, it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

More specifically, tracheal tubes may be used to control the flow of air or other gases through a patient's trachea. Such tracheal tubes may include endotracheal tubes or tracheostomy tubes. While patients may be intubated using endotracheal tubes during emergencies or shorter hospital stays, tracheostomy tubes are typically used for prolonged ventilation, as the use of a tracheostomy tube may be more comfortable for a patient.

A typical tracheostomy tube is generally inserted into the trachea via a stoma, which is a surgical incision in the neck. After insertion of the tube into the trachea, a portion of the tracheostomy tube remains outside the patient. This portion extends outwards from the neck and may connect the tracheostomy tube to a ventilator or other medical device. Generally, this exterior portion of the tube is held in place by a flange that rests on the patient's neck and is further secured by straps to the patient. The inserted portion of the tracheostomy tube is generally mechanically coupled to the flange, typically by a snap or screw mechanism or bonding on the underside of the flange, which rests on the patient's neck. During prolonged intubation periods, this mechanical connection point may cause irritation at the stoma site, thereby causing discomfort for the patient. Additionally, the portions of the flange that contact the patient's neck are often rigid, and, accordingly, when such portions rub against the patient's neck during movement of the patient, the patient may experience additional discomfort.

Certain devices attempt to address these problems by providing pillow or fabric protectors for the neck that may lift the flange slightly off the neck to avoid skin irritation, providing some additional comfort. However, these devices may be a less stable base for the tube at the patient's neck. Additionally, these devices often need to be replaced during periods of prolonged intubation, and it can be difficult for a medical practitioner to replace these devices since they are typically located between the flange and the patient's neck. Still further, during replacement, when the exterior portion of the tube (e.g., the flange) is shifted or moved, these movements may be translated to the interior potion of the tube. These movements may cause some additional discomfort for the patient if the tracheostomy tube shifts position within the trachea. Accordingly, there exists a need for tracheostomy tubes that overcome these drawbacks.

### BRIEF DESCRIPTION OF THE DRAWINGS

Advantages of the disclosed techniques may become apparent upon reading the following detailed description and upon reference to the drawings in which:

FIG. 1 is a perspective view of a tracheostomy tube including interior and exterior flanges having variable volumes according to an embodiment;

FIG. 2 illustrates an embodiment of a tracheostomy tube positioned within a patient's trachea and having an inflatable exterior flange;

FIG. 3 illustrates an embodiment of a tracheostomy tube positioned within a patient's trachea and having inflatable interior and exterior flanges;

FIG. 4 is a flow chart illustrating a method of using the tracheostomy tube of FIG. 3 according to an embodiment;

FIG. 5 illustrates an embodiment of an inflatable flange mounted on a tubular body and having a first inflatable portion and a second inflatable portion; and

FIG. 6 illustrates an embodiment of an inflatable flange mounted on a tubular body and having apertures suitable for receiving securement straps.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, provided herein are tracheostomy tube assemblies including one or more flange members having a variable volume. For example, in presently contemplated embodiments, the flange member may be inflatable and capable of engaging a patient's neck to maintain a portion of the tracheostomy tube assembly outside of the patient's body. As compared to traditional designs, such tube assemblies may enable increased patient comfort during use. For example, as compared to conventional flanges that are often made of a relatively hard, inflexible material, the provided inflatable flanges may better conform to the patient's anatomy, possibly reducing or preventing the likelihood of discomfort at the stoma site. The foregoing feature may increase patient comfort since the allowed movement of the inflatable flange member, which rests against the patient's neck, may more closely follow patient movement as compared to traditional inflexible designs.

The provided tracheostomy tube assemblies may be disposable rather than reusable and may be capable of conveying gas to and from the patient, such as during medical situations that necessitate prolonged ventilation. As such, the devices and techniques provided herein may enable maintaining a bidirectional gas flow between the patient and an external ventilation device. Accordingly, the tracheostomy tube assemblies provided herein may be adapted to be inserted into the trachea via a surgical incision in the neck such that after insertion of the tube into the trachea, a portion of the tube remains outside the patient. This portion extends outwards from the neck and may connect the tracheostomy tube to a ventilator or other medical device. That is, the provided tracheostomy tube assemblies may be used in conjunction with auxiliary devices, such as airway accessories, ventilators, humidifiers, and so forth, which may cooperate with the tube assemblies to maintain airflow to and from the lungs of the patient. For example, the tracheal tubes may be coupled to an adapter or connector that is configured to couple the tracheostomy tube assemblies described herein to the desired auxiliary device.

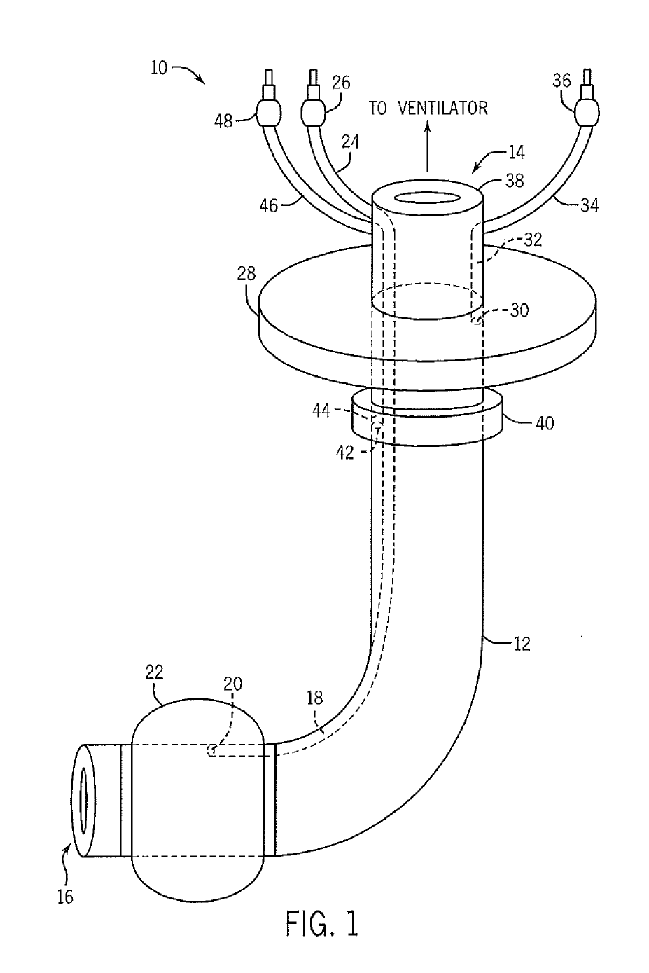

Turning now to the drawings, FIG. 1 is a perspective view of an exemplary tracheostomy tube assembly 10 according to an embodiment. In the depicted embodiment, the tracheostomy tube assembly 10 includes an arcuate cannula 12 having a proximal end 14 and a distal end 16, which is generally sized and configured to be inserted into a patient's neck through a surgical incision for prolonged ventilation. When the tracheostomy tube assembly 10 is in use, the distal end 16 as well as the major portion of the length of the cannula 12 will reside within the trachea, with the proximal end 14 being generally flush with the anterior surface of the patient's neck. The cannula 12 may also feature a lumen 18 within the wall, terminating in notch 20 that may be used to fill a balloon type sealing cuff 22 at the patient insertion end. In some embodiments, the cuff 22 may be a urethane balloon bonded to the exterior of the cannula 12 such that the notch 20 is encompassed. In certain embodiments, the cuff 22 may be inflated within the patient's airway, for example, via inflation tube 24 and inflation assembly 26, to provide an additional seal. However, in other embodiments, the cannula 12 may be provided without the cuff 22.

In some embodiments, the cannula 12 may also include a suction lumen (not shown in FIG. 1) that extends from a location on the proximal end 14 of the cannula 12 positioned outside the body when in use to a location around the cuff 22 inside the body. The suction lumen may terminate in a port through which secretions accumulated around the cuff may be aspirated. For example, a port may be located above the cuff 22 or one or more ports may be located anywhere along the length of the cannula 12 such that they aspirate secretions from the airway of the patient. Further, in some embodiments, an exterior suction tube may connect to the suction lumen for the removal of the suctioned fluids, for example, via a vacuum connected to the exterior suction tube.

The tracheostomy tube assembly 10 also features a flange 28 having a variable volume and being connected to the proximal end 14 of the cannula 12. In the depicted embodiment, the flange 28 is an inflatable flange fluidly accessible via notch 30 coupled to inflation lumen 32. This lumen 32 is an airway that may be used to fill the inflatable flange 28. That is, during use, a medical practitioner may inject a suitable fluid, such as air, into the lumen 32 via inflation tube 34 and inflation assembly 36, thereby transferring the air to the inflatable flange 28. In this manner, the flange 28 may be inflated to a desired volume, which may be determined by the medical professional based on factors such as the patient's size, the tracheostomy tube size, and so forth. When inflated, the flange 28 is designed to rest on the neck of the patient to maintain a portion of the tracheostomy tube assembly 10 outside of the patient's body.

Features of the flange 28 having a variable volume and being illustrated in FIG. 1 may offer distinct advantages over traditional designs. For example, the variable volume flange 28 may be formed from materials having desirable mechanical properties (e.g., puncture resistance, pin hole resistance, tensile strength, and so forth) and desirable chemical properties (e.g., biocompatibility). In certain embodiments, it may be desirable for the flange 28 to be relatively soft as compared to traditional rigid flanges, thus being capable of compressing and expanding to conform to the patient's anatomy. This feature may enable increased patient comfort as compared to traditional rigid flange designs.

In embodiments in which the variable volume flange is an inflatable flange, the walls of the flange 28 may be made of a polyurethane (e.g., Dow Pellethane® 2363-80A) having suitable mechanical and chemical properties. In other embodiments, the walls of the flange 28 may be made of silicone or a suitable polyvinyl chloride (PVC). In certain embodiments, the inflatable flange 28 may be generally sized and shaped as a high volume, low pressure cuff that may be designed to be inflated to pressures between approximately 15 cm H₂O and 30 cm H₂O. Additionally, in some embodiments, the wall thickness of the inflatable flange 28 may vary depending on the intended use of the tracheostomy tube assembly 10. Still further, in some embodiments, the flange 28 may not be inflatable, but instead, the flange 28 may be made of a material, such as foam, that is capable of expanding and contracting to various volumes. Additionally, it should be noted that the flange 28 may take on a variety of suitable shapes and sizes, not limited to the shapes of the depicted embodiments.

Additionally, the flange 28 may feature a conduit 38 that is substantially in-line with the proximal end 14 of the cannula 12. Generally, the conduit 38 may be adapted to connect the tracheostomy tube assembly 10 to any suitable medical device. For example, in certain embodiments, the conduit 38 may serve as an insertion point for a disposable cannula lining or may be suitably sized and shaped to connect the tracheostomy tube assembly 10 via medical tubing or other devices to a mechanical ventilator.

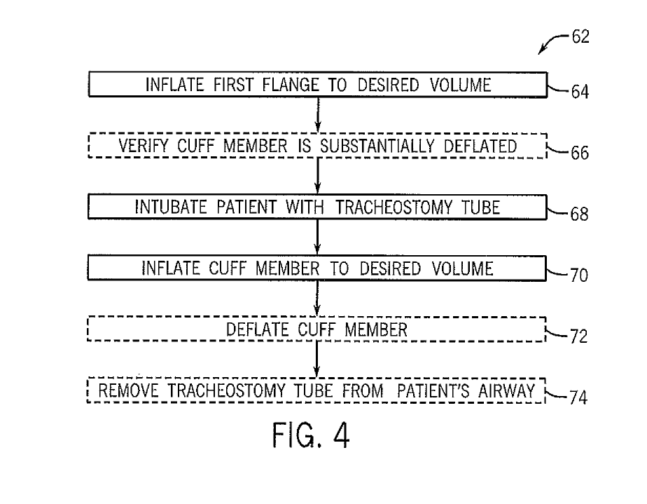

In the depicted embodiment, the tracheostomy tube assembly 10 also includes an inflatable cuff member 40 disposed about the cannula 12. During use, the cuff member 40 is adapted to be inserted into the patient's trachea and to remain within the patient's airway throughout the period of ventilation of the patient. More specifically, the cuff member 40 may be inserted into the patient's airway in a deflated state and subsequently inflated when the tracheostomy tube assembly 10 is appropriately positioned. To that end, a notch 42 coupled to an inflation lumen 44, an inflation tube 46, and an inflation assembly 48 enable a user to insert air into the cuff member 40 to inflate the cuff member 40 to the desired volume within the patient's trachea. In the depicted embodiment, the inflatable flange 28 and the cuff member 40 are designed to be inflated on opposite sides of the stoma, thereby securing the tracheostomy tube assembly 10 in the desired position, as discussed in more detail below with respect to FIGS. 3 and 4. To that end, in certain embodiments, the cuff member 40 may be designed as a low pressure cuff that may be inflated to pressures between approximately 15 μm H₂O and 30 cm H₂O. Further, it should be noted that the cuff member 40 may take on a variety of suitable shapes and sizes, not limited to the shapes of the depicted embodiments.

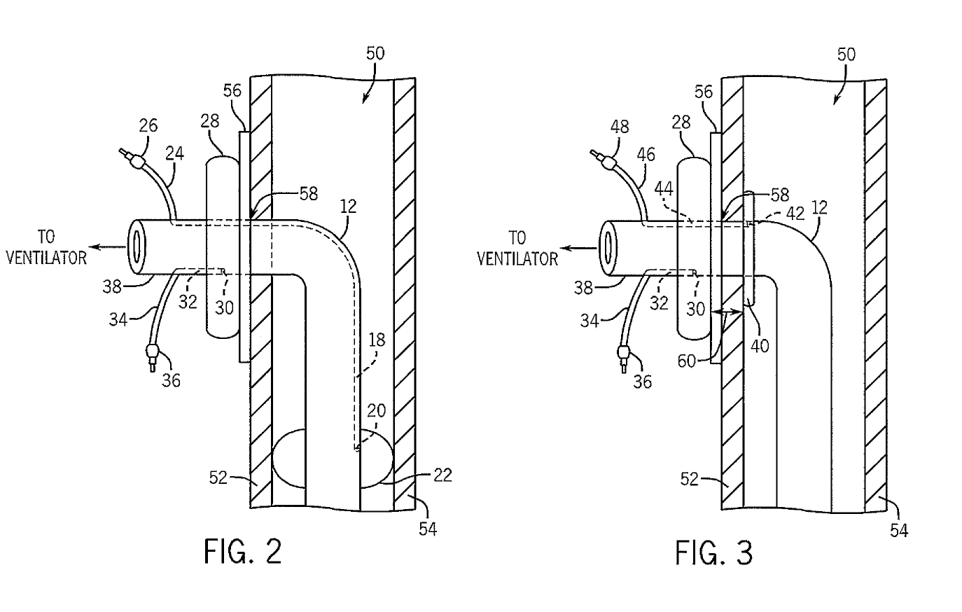

FIGS. 2 and 3 illustrate side views of embodiments of tracheostomy tube assemblies positioned within a trachea 50 of a patient in accordance with presently contemplated embodiments. It should be noted that although FIGS. 2 and 3 illustrate the patient's anatomy as a trachea 50 having tracheal walls 52 and 54, as understood by those skilled in the art, the anatomy of a patient present during use may include other features, such as additional body tissues (e.g., skin, body fat, etc.). Indeed, the illustrations shown in FIGS. 2 and 3 are merely schematics shown for illustrative purposes.

Turning now to FIG. 2, in the depicted embodiment, the cannula 12 illustrates the cuff 22 in its inflated state within the trachea 50. Accordingly, as shown, the inflated cuff 22 contacts the tracheal walls 52 and 54 to secure the tracheostomy tube assembly in the desired location within the trachea 50. As noted above, during insertion, the cuff 22 may be deflated, and, once positioned, the cuff 22 may be inflated to the desired volume.

Likewise, the inflatable flange 28 is illustrated in an inflated state, for example, after the medical practitioner has inflated the flange 28 to the desired volume for the given application. Still further, in the depicted embodiment, a stoma pad 56 is positioned between the flange 28 and the tracheal wall 52 to decrease patient discomfort due to irritation of the stoma. In certain embodiments, the inflatable flange 28 may offer advantages over rigid flanges having a predetermined volume because the inflatable flange 28 may be deflated and re-inflated during the intubation period as desired. For example, in instances of prolonged ventilation, it may be desirable to remove and replace the stoma pad 56. By varying the volume of the flange 28 between a fully inflated state and a partially or completely deflated state, the stoma pad 56 may be more easily removed and replaced with a reduced level of discomfort experienced by the patient.

FIG. 3 similarly illustrates a side view of another embodiment of a tracheostomy tube assembly positioned within the patient's trachea 50. In this embodiment, however, the cannula 12 does not include the cuff 22 mounted thereon, but rather, the cuff member 40 is provided. In the depicted view, the tracheostomy tube assembly is positioned within the trachea 50 in such a way that is suitable for a period of prolonged ventilation. As such, the inflatable flange 28 and the cuff member 40 are inflated to their respective desired volumes. When inflated in this manner, a distance 60 between the flange 28 and the cuff member 40 along the length of the cannula 12 is established. In some embodiments, this distance 60 may vary depending on the type of the tracheostomy tube assembly. For example, a pediatric assembly may have a smaller flange to cuff distance than an adult tube assembly.

Still further, when inflated and positioned as illustrated, the flange 28 exerts pressure on the stoma pad 56, and the cuff member 40 exerts pressure on the tracheal wall 52. In this manner, the stoma pad 56 and the tracheal wall 52 become compressed between the flange 28 and the cuff member 40, and the tracheostomy tube assembly becomes secured in the patient's trachea. The foregoing feature may reduce or eliminate the desirability of including the cuff 22 on the cannula 12 in some embodiments. However, if desired, the cuff 22 may also be provided to center or otherwise position the cannula 12 within the trachea 50.

Still further, it should be noted that although in the illustrated embodiments, the cuff member 40 is coupled to a cannula that includes the inflatable flange member 28, in other embodiments, the cuff member 40 may be utilized in conjunction with other variable or constant volume flanges. For example, in one embodiment, the cuff member 40 may be utilized with a traditional rigid flange member. In these embodiments, the stoma pad 56 and the tracheal wall 52 may be compressed between the conventional, rigid flange member and the cuff member 40 to secure the tracheostomy tube assembly in the patient.

To facilitate insertion of the cannula 12 into the patient's trachea 50, it may be desirable to maintain the cuff member 40 in a deflated position until the cannula 12 is advanced beyond the stoma site 58. FIG. 4 illustrates an embodiment of a method 62 that may be employed by a medical practitioner to utilize the tracheostomy tube assembly of FIG. 3. The depicted method 62 includes the step of inflating the first flange (e.g., flange 28) to the desired volume (block 64). In certain embodiments, the first flange may be the flange that is designed to rest against the patient's neck and maintain a portion of the tracheostomy tube assembly outside of the patient's trachea.

In some embodiments, the medical practitioner may verify that the cuff member (e.g., cuff member 40) is substantially deflated (block 66). Again, the cuff member may be designed for positioning within the patient's trachea when the cannula is in its fully inserted position. The patient may then be intubated with the tracheostomy tube assembly having the cuff member in a deflated state (block 68). Once the cannula is inserted to the desired position, the cuff member is inflated to the desired volume (block 70), and the tracheal wall becomes compressed between the exteriorly located flange and the interiorly located cuff member. If the medical practitioner desires to remove the tracheostomy tube assembly from the patient's trachea, the cuff member may be deflated (block 72), and the tracheostomy tube may be removed from the patient's airway (block 74).

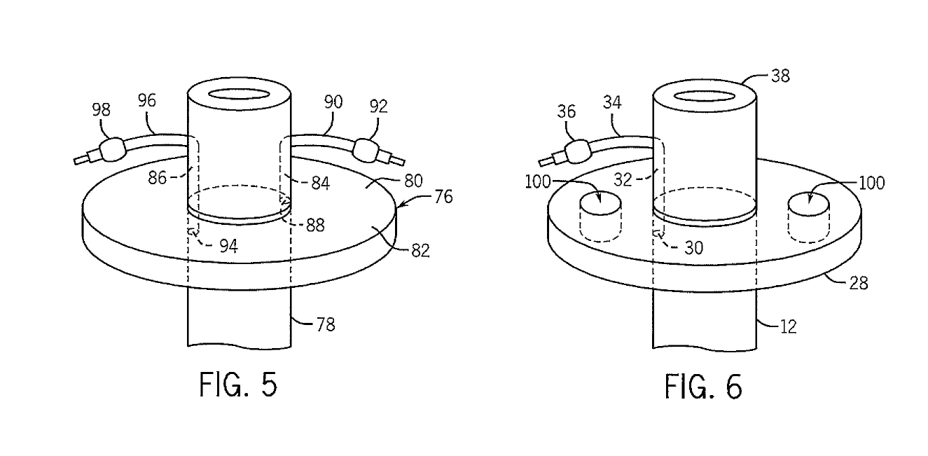

FIG. 5 illustrates an embodiment of a segmented inflatable flange 76 mounted on a tubular body 78. In the depicted embodiment, the segmented inflatable flange 76 includes a first inflatable region 80 and a second inflatable region 82. That is, in some embodiments, the flange 76 may include two or more inflatable regions adapted to be separately inflated and deflated, as desired by the user. As such, the pressure of each inflatable region may be separately adjusted by an operator. Further, in particular embodiments, the inflatable regions may have different inflation capacities, and, when fully inflated, the inflatable regions may position the tubular body 78 in the desired location within the patient's airway.

To enable independent inflation of each of the inflatable regions 80 and 82, inflation lumens 84 and 86 are provided. Specifically, a user may insert air into inflatable region 80 via notch 88, inflation lumen 84, inflation tube 90, and inflation assembly 92. Similarly, a user may insert air into inflatable region 82 via notch 94, inflation lumen 86, inflation tube 96, and inflation assembly 98. As such, the two inflation regions 80 and 82 may be inflated or deflated to the desired volumes.

It should be noted that based on the intended use of the tracheostomy tube assembly, a different arrangement or quantity of inflatable regions may be provided. The inflatable flanges and/or cuff members of presently disclosed embodiments may take on various forms having a variety of arrangements of inflatable regions. For example, embodiments of the inflatable flanges and/or cuff members may have any combination of any quantity of vertically, horizontally, or angularly disposed inflatable regions, such as regions arranged in a honeycomb structure, among other desirable structures.

FIG. 6 illustrates an alternate embodiment of the flange 28 mounted on the cannula 12 and including features that facilitate attachment of the tracheostomy tube assembly to the patient's neck. For example, in the depicted embodiment, the flange 28 includes apertures 100 designed to accommodate attachment straps that may secure the tracheostomy tube assembly to the patient's neck. It should be noted that in this embodiment, the flange 28 may be inflatable as previously described, but also may be made of a non-inflatable material, such as foam, having a variable volume.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A tracheostomy tube assembly, comprising:
a cannula comprising a distal end and a proximal end, the distal end being configured to be inserted into a patient's trachea; and
a flange member disposed about the proximal end of the cannula, wherein the flange member is configured to expand to a first volume and to contract to a second volume, the first volume being greater than the second volume.

2. The tracheostomy tube assembly of claim 1, comprising a conduit disposed on the proximal end of the cannula and being configured to operatively connect to a medical device.

3. The tracheostomy tube assembly of claim 2, wherein the medical device comprises an airway accessory, a ventilator, a humidifier, or a combination thereof.

4. The tracheostomy tube assembly of claim 1, wherein the flange member is an inflatable flange member configured to be inflated to the first volume and to be deflated to the second volume.

5. The tracheostomy tube assembly of claim 4, wherein the flange member is made of a flexible polyvinyl chloride (PVC).

6. The tracheostomy tube assembly of claim 4, wherein the flange member comprises more than one inflatable chamber, and wherein each of the more than one inflatable chambers are configured to be independently inflated and deflated.

7. The tracheostomy tube assembly of claim 1, comprising a cuff member disposed about the cannula on a portion of the cannula that is configured to be disposed within the patient's trachea, wherein the cuff member is configured to be inflated and deflated.

8. The tracheostomy tube assembly of claim 1, comprising a cuff disposed about the distal end of the cannula and configured to be inflated to seal against walls of the patient's trachea.

9. The tracheostomy tube assembly of claim 1, wherein the flange member comprises a pair of apertures configured to receive securement straps adapted to secure the tracheostomy tube assembly to the patient's neck.

10. A tracheostomy tube assembly, comprising:
a cannula comprising a distal end and a proximal end, the distal end being configured to be inserted into a patient's trachea;
a flange member disposed about the proximal end of the cannula, wherein the flange member is configured to engage the patient's neck to maintain a portion of the tracheostomy tube outside of the patient's trachea; and
an inflatable cuff member disposed about the proximal end of the cannula and configured to be inflated and deflated to seal against a wall of the patient's trachea in a location of a stoma site to compress the wall of the patient's trachea between the inflatable cuff member and the flange member.

11. The tracheostomy tube assembly of claim 10, wherein the flange member is a variable volume flange member.

12. The tracheostomy tube assembly of claim 11, wherein the flange member is an inflatable flange member configured to be inflated and deflated to engage and disengage with the patient's neck.

13. The tracheostomy tube assembly of claim 12, wherein the flange member is a segmented flange member comprises a first inflatable chamber and a second inflatable chamber configured to be independently inflated.

14. The tracheostomy tube assembly of claim 12, comprising an inflation lumen disposed in a wall of the cannula and configured to facilitate the transfer of air to and from the flange member for inflation and deflation.

15. The tracheostomy tube assembly of claim 10, wherein the flange member comprises a pair of apertures configured to receive securement straps adapted to secure the tracheostomy tube assembly to the patient's neck.

16. A method of sealing a patient's trachea, comprising:
inserting a tracheostomy tube into a patient's trachea, wherein the tracheostomy tube comprises:
a cannula comprising a distal end and a proximal end, the distal end being configured to be inserted into a patient's trachea; and
an inflatable flange member disposed about the proximal end of the cannula, wherein the inflatable flange member is configured to be inflated and deflated and to engage the patient's neck to maintain the inflatable flange member and a portion of the cannula outside of the patient's trachea.

17. The method of claim 16, comprising inflating the inflatable flange member to a predetermined volume before inserting the tracheostomy tube into the patient's trachea.

18. The method of claim 16, wherein the tracheostomy tube comprises an inflatable cuff member disposed about the proximal end of the cannula and configured to be inflated and deflated to seal against a wall of the patient's trachea in a location of a stoma site.

19. The method of claim 18, comprising inflating the cuff member to compress a portion of the wall of the patient's trachea at the stoma site between the inflatable cuff member and the inflatable flange member.

20. The method of claim 16, wherein the tracheostomy tube comprises a cuff disposed about the distal portion of the cannula and configured to be inflated to seal against the walls of the patient's trachea.
